(19) United States
(12) Patent Application Publication
(10) Pub. No.: US 2025/0044446 A1
(43) Pub. Date: Feb. 6, 2025

(54) METHODS TO MAINTAIN IMAGE QUALITY IN ULTRASOUND IMAGING AT REDUCED COST, SIZE, AND POWER

(71) Applicant: Exo Imaging, Inc., Santa Clara, CA (US)

(72) Inventors: Miles WERNICK, Evanston, IL (US); Sandeep AKKARAJU, Wellesley, MA (US); Drake GUENTHER, Hillsborough, CA (US); Kutay ÜSTÜNER, Mountain View, CA (US); Yusuf HAQUE, Woodside, CA (US)

(21) Appl. No.: 18/799,921

(22) Filed: Aug. 9, 2024

Related U.S. Application Data
(63) Continuation of application No. 17/272,901, filed on Mar. 2, 2021, now Pat. No. 12,061,257.
(60) Provisional application No. 62/785,315, filed on Dec. 27, 2018.

(51) Int. Cl. G01S 15/89; G01S 7/52

(52) U.S. Cl. CPC G01S 15/8977; G01S 7/52077; G01S 7/52085; G01S 7/52096; G01S 15/8979

(57) ABSTRACT

Computer-implemented medical ultrasound imaging methods and systems for performing the methods can be performed using a plurality of frames of an ultrasound image sequence that each having a high image quality to improve the image quality of lower-image-quality frame of the ultrasound image sequence.

### CROSS-REFERENCE TO RELATED APPLICATIONS

This application is a continuation of U.S. patent application Ser. No. 17/272,901, filed Mar. 2, 2021, which is the National Stage entry of International Application No. PCT/US2019/068004, filed Dec. 20, 2019, which claims the benefit of U.S. Provisional Application No. 62/785,315, filed Dec. 27, 2018, each of which are herein incorporated by reference in their entireties.

### BACKGROUND

Ultrasound imaging is widely used in the fields of medicine and non-destructive testing.

### SUMMARY

Typical ultrasound imaging devices are bulky and costly, and there exists a need for small, portable, and low-cost devices. Portability of an ultrasound imaging device demands that its electrical power requirements be minimized.

Cost, size and power consumption of an ultrasound imaging device can be reduced through various modifications of the process by which the ultrasound pulses are produced and sensed. The modifications to reduce power consumption include reducing active channel count, front-end gain and bias, analog-to-digital conversion (ADC) sampling rate, lateral sampling rate, temporal sampling rate, number of parallel receive beams, beam-bandwidth product, filter kernel lengths, filter coefficient quantization levels, data rate and word length, ensemble length for Doppler, etc. However, such modifications generally result in reduced image quality in the form of a combination of reduced spatial resolution, contrast resolution, temporal resolution, sensitivity, penetration, and dynamic range, or increased imaging and spectral artifacts. There is a perceived risk that current methodologies may fail to accurately represent subtle image details that convey important diagnostic information.

There exists a need to counteract these reductions in image quality so that good image quality can be achieved with low cost, low performance, small form factor integrated circuits and processors at reduced levels of power consumption. Accordingly, the subject matter described herein, in some embodiments, uses machine learning to permit ultrasound images to be produced with lower cost, lower power and smaller form factor devices without the associated loss of image quality. In some embodiments, the subject matter described herein makes use of high-quality and reduced-quality frames. In further embodiments, the subject matter described herein makes alternating use of high-quality and reduced-quality frames. In so doing, the subject matter described herein, in such embodiments, incorporates high-quality images periodically during processing to minimize risk of failing to accurately represent subtle image details by ensuring that high-quality image details are available to the model.

In one aspect, disclosed herein are computer-implemented medical ultrasound imaging methods comprising: forming a first frame of an ultrasound image sequence with high image quality; forming at least one frame of the ultrasound image sequence with reduced image quality; forming a second frame of the ultrasound image sequence with high image quality; and improving the quality of the at least one frame of the ultrasound image sequence with reduced image quality using the first or the second frames of the ultrasound image sequence with high quality. In some embodiments, the quality of the at least one frame of the ultrasound image sequence with reduced image quality is improved by using the first and the second frame of the ultrasound image sequence with high quality. In some embodiments, the method further comprises improving the quality of the first frame of the ultrasound image sequence with high image quality or the second frame of the ultrasound image sequence with high image quality. In various embodiments, the image quality comprises spatial resolution, contrast resolution, signal-to-noise ratio, and/or signal dynamic range. In some embodiments, each frame is a 2-dimensional image. In other embodiments, each frame is a 3-dimensional image. In some embodiments, each frame is a B-Mode image. In other embodiments, each frame is a color Doppler image. In yet other embodiments, each frame is a spectral Doppler strip. In some embodiments, the improving the quality of the at least one frame of the ultrasound image sequence with reduced image quality is achieved by application of machine learning. In some embodiments, the reduced image quality is at least partially the result of low spatial sampling incurred by using a reduced number of transducer elements. In some embodiments, the reduced image quality is at least partially the result of low temporal sampling incurred by using a low temporal sampling rate. In some embodiments, the reduced image quality is at least partially the result of low spatial frequency sampling. In some embodiments, the reduced image quality is at least partially the result of temporal delay quantization used during the beamforming process. In some embodiments, the reduced image quality is at least partially due to not performing phase aberration correction. In some embodiments, the reduced image quality is at least partially due to not performing aperture coherence function-based imaging techniques. In some embodiments, the reduced image quality is at least partially due to a reduced number of transmissions (line spacing and ensemble length). In various embodiments, the reduced image quality is due to a combination of two or more techniques used to reduce image quality selected from the list consisting of: low spatial sampling incurred by using a reduced number of elements of the ultrasound transducer, low temporal sampling incurred by using a low temporal sampling rate, low spatial frequency sampling, temporal delay quantization used during a beamforming process, not performing phase aberration correction, not performing aperture coherence function-based imaging techniques, and sending a reduced number of transmissions (line spacing and ensemble length). In some embodiments, the frames are formed using a transducer, and wherein the transducer comprises a pMUT device. In some embodiments, the frames are formed using a transducer and the transducer comprises a 1.25D, 1.5D, 1.75D, or 2D array.

In another aspect, disclosed herein are medical ultrasound imaging systems comprising: a medical ultrasound imaging device comprising an ultrasound transducer; and at least one processor; the system configured to perform functions comprising: forming, using the ultrasound transducer, a first frame of an ultrasound image sequence with high image quality; forming, using the ultrasound transducer, at least one frame of the ultrasound image sequence with reduced image quality; forming, using the ultrasound transducer, a second frame of the ultrasound image sequence with high image quality; and improving, using the at least one processor, the quality of the at least one frame of the ultrasound image sequence with reduced image quality using the first or the second frames of the ultrasound image sequence with high quality. In some embodiments, the quality of the at least one frame of the ultrasound image sequence with reduced image quality is improved by using the first and the second frame of the ultrasound image sequence with high quality. In some embodiments, the system is further configured to perform functions comprising: improving the quality of the first frame of the ultrasound image sequence with high image quality or the second frame of the ultrasound image sequence with high image quality. In various embodiments, image quality comprises spatial resolution, contrast resolution, signal-to-noise ratio, and/or signal dynamic range. In some embodiments, each frame is a 2-dimensional image. In other embodiments, each frame is a 3-dimensional image. In some embodiments, each frame is a B-Mode image. In some embodiments, each frame is a color Doppler image. In some embodiments, each frame is a spectral Doppler strip. In some embodiments, the improving the quality of the at least one frame of the ultrasound image sequence with reduced image quality is achieved by application of machine learning. In some embodiments, the reduced image quality is the result of low spatial sampling incurred by using a reduced number of elements of the ultrasound transducer. In some embodiments, the reduced image quality is the result of low temporal sampling incurred by using a low temporal sampling rate. In some embodiments, the reduced image quality is the result of low spatial frequency sampling. In some embodiments, the reduced image quality is the result of temporal delay quantization used during a beamforming process. In some embodiments, the reduced image quality is due to not performing phase aberration correction. In some embodiments, the reduced image quality is due to not performing aperture coherence function-based imaging techniques. In some embodiments, the reduced image quality is due to a reduced number of transmissions (line spacing and ensemble length). In various embodiments, the reduced image quality is due to a combination of two or more techniques used to reduce image quality selected from the list consisting of: low spatial sampling incurred by using a reduced number of elements of the ultrasound transducer, low temporal sampling incurred by using a low temporal sampling rate, low spatial frequency sampling, temporal delay quantization used during a beamforming process, not performing phase aberration correction, not performing aperture coherence function-based imaging techniques, and sending a reduced number of transmissions (line spacing and ensemble length). In some embodiments, the ultrasound transducer comprises a pMUT device. In some embodiments, the ultrasound transducer comprises a 1.25D, 1.5D, 1.75D, or 2D array of elements. In some embodiments, the at least one processor comprises an application-specific integrated circuit (ASIC). In some embodiments, the at least one processor comprises a mobile computing device in communication with the medical ultrasound imaging device.

### BRIEF DESCRIPTION OF THE DRAWINGS

A better understanding of the features and advantages of the present subject matter will be obtained by reference to the following detailed description that sets forth illustrative embodiments and the accompanying drawings of which:

FIG. 1 illustrates two scan procedures: in Scan Procedure 1, every Nth frame is a High-Quality Image and all the other frames are Reduced-Quality Images; and in Scan Procedure 2, all the frames are Reduced-Quality Images;

FIG. 2A illustrates a spatio-temporal prediction Model comprising Conv and BC-LSTM modules (ReLU indicates a rectified linear unit); and

FIG. 2B illustrates the internal structure of a BC-LSTM module of FIG. 2A.

### DETAILED DESCRIPTION

Disclosed herein, in some embodiments, are computer-implemented medical ultrasound imaging methods comprising: forming a first frame of an ultrasound image sequence with high image quality; forming at least one frame of the ultrasound image sequence with reduced image quality; forming a second frame of the ultrasound image sequence with high image quality; and improving the quality of the at least one frame of the ultrasound image sequence with reduced image quality using the first or the second frames of the ultrasound image sequence with high quality. In some embodiments, the quality of the at least one frame of the ultrasound image sequence with reduced image quality is improved by using the first and the second frame of the ultrasound image sequence with high quality. In some embodiments, the method further comprises improving the quality of the first frame of the ultrasound image sequence with high image quality or the second frame of the ultrasound image sequence with high image quality. In some embodiments, the image quality comprises spatial resolution. In some embodiments, the image quality comprises contrast resolution. In some embodiments, the image quality comprises signal-to-noise ratio. In some embodiments, the image quality comprises signal dynamic range. In some embodiments, each frame is a 2-dimensional image. In other embodiments, each frame is a 3-dimensional image. In some embodiments, each frame is a B-Mode image. In other embodiments, each frame is a color Doppler image. In yet other embodiments, each frame is a spectral Doppler strip. In some embodiments, the improving the quality of the at least one frame of the ultrasound image sequence with reduced image quality is achieved by application of machine learning. In some embodiments, the reduced image quality is at least partially the result of low spatial sampling incurred by using a reduced number of transducer elements. In some embodiments, the reduced image quality is at least partially the result of low temporal sampling incurred by using a low temporal sampling rate. In some embodiments, the reduced image quality is at least partially the result of low spatial frequency sampling. In some embodiments, the reduced image quality is at least partially the result of temporal delay quantization used during the beamforming process. In some embodiments, the reduced image quality is at least partially due to not performing phase aberration correction. In some embodiments, the reduced image quality is at least partially due to not performing aperture coherence function-based imaging techniques. In some embodiments, the reduced image quality is at least partially due to a reduced number of transmissions (line spacing and ensemble length). In some embodiments, the reduced image quality is at least partially due to a combination of two or more of the above techniques used to reduce image quality. In some embodiments, the frames are formed using a transducer, and wherein the transducer used is a pMUT device. In some embodiments, the frames are formed using a transducer, and wherein the transducer used is a 1.25D, 1.5D, 1.75D, or 2D array.

Also, disclosed herein, in some embodiments, are medical ultrasound imaging systems comprising: a medical ultrasound imaging device comprising an ultrasound transducer; and at least one processor; the system configured to perform functions comprising: forming, using the ultrasound transducer, a first frame of an ultrasound image sequence with high image quality; forming, using the ultrasound transducer, at least one frame of the ultrasound image sequence with reduced image quality; forming, using the ultrasound transducer, a second frame of the ultrasound image sequence with high image quality; and improving, using the at least one processor, the quality of the at least one frame of the ultrasound image sequence with reduced image quality using the first or the second frames of the ultrasound image sequence with high quality. In some embodiments, the quality of the at least one frame of the ultrasound image sequence with reduced image quality is improved by using the first and the second frame of the ultrasound image sequence with high quality. In some embodiments, the device is further configured to perform functions comprising: improving the quality of the first frame of the ultrasound image sequence with high image quality or the second frame of the ultrasound image sequence with high image quality. In various embodiments, image quality comprises spatial resolution, contrast resolution, signal-to-noise ratio, and/or signal dynamic range. In some embodiments, each frame is a 2-dimensional image. In other embodiments, each frame is a 3-dimensional image. In some embodiments, each frame is a B-Mode image. In some embodiments, each frame is a color Doppler image. In some embodiments, each frame is a spectral Doppler strip. In some embodiments, the improving the quality of the at least one frame of the ultrasound image sequence with reduced image quality is achieved by application of machine learning. In some embodiments, the reduced image quality is the result of low spatial sampling incurred by using a reduced number of elements of the ultrasound transducer. In some embodiments, the reduced image quality is the result of low temporal sampling incurred by using a low temporal sampling rate. In some embodiments, the reduced image quality is the result of low spatial frequency sampling. In some embodiments, the reduced image quality is the result of temporal delay quantization used during a beamforming process. In some embodiments, the reduced image quality is due to not performing phase aberration correction. In some embodiments, the reduced image quality is due to not performing aperture coherence function-based imaging techniques. In some embodiments, the reduced image quality is due to a reduced number of transmissions (line spacing and ensemble length). In various embodiments, the reduced image quality is due to a combination of two or more techniques used to reduce image quality selected from the list consisting of: low spatial sampling incurred by using a reduced number of elements of the ultrasound transducer, low temporal sampling incurred by using a low temporal sampling rate, low spatial frequency sampling, temporal delay quantization used during a beamforming process, not performing phase aberration correction, not performing aperture coherence function-based imaging techniques, and sending a reduced number of transmissions (line spacing and ensemble length). In some embodiments, the ultrasound transducer comprises a pMUT device. In some embodiments, the ultrasound transducer comprises a 1.25D, 1.5D, 1.75D, or 2D array of elements. In some embodiments, the at least one processor comprises an application-specific integrated circuit (ASIC).

### Machine Learning Terms

A machine learning algorithm (“Model”) is a mathematical function ƒ(x; w) in which x (“Input”) is a vector (list) of mathematical variables, y (“Label”) is a vector of mathematical variables, and w (“Parameter Vector”) is a vector of mathematical parameters of the Model. The Model is designed by choosing a value for the Parameter Vector based on a multiplicity of pairs (“Training Set”) {xᵢ, yᵢ}, i=1, . . . , P, where xᵢ and yᵢ are known corresponding example values for x and y, respectively. Let ŷᵢ denote the value produced by substitution of xᵢ for x in the Model. The Parameter Vector is chosen via a mathematical optimization procedure such that, in general, a value ŷᵢ (“Predicted Label”) closely approximates the corresponding value yᵢ (“True Label”). The Model is said to predict the Label based on the Input. A vector that represents the Input or Label can describe an image if its elements are the pixel values of an image arranged in lexicographic order. A vector that represents the Input or Label can alternatively describe a multiplicity of images by concatenation of vectors representing the individual images to form the vector.

### Certain Definitions

Unless otherwise defined, all technical terms used herein have the same meaning as commonly understood by one of ordinary skill in the art to which the present subject matter belongs. As used in this specification and the appended claims, the singular forms “a,” “an,” and “the” include plural references unless the context clearly dictates otherwise. Any reference to “or” herein is intended to encompass “and/or” unless otherwise stated.

As used herein, a “High-Quality Image” is a frame of an ultrasound image sequence in which the data acquisition parameters have been chosen in such a way as to produce acceptably high image quality in a manner that would be feasible in actual operation of the device in its intended application.

As used herein, a “Reduced-Quality Image” is a frame of an ultrasound image sequence in which, at the expense of reduced image quality, the data acquisition parameters have been deliberately chosen to reduce power consumption, size or cost of the device relative to that required to obtain a High-Quality Image.

As used herein, a “Restored Image” is a frame of an ultrasound image sequence obtained by applying a Model to one or more Reduced-Quality Images to counteract the loss of image quality in the Reduced-Quality Images, thereby restoring image quality to approximate that of a High-Quality Image. A Restored Image comprises a Predicted Label produced by the Model.

As used herein, an “Enhanced-Quality Image” is a frame of an ultrasound image sequence, created solely for purposes of training the Model, which is obtained by applying techniques and design parameters that would be infeasible in actual operation of the imaging device in its intended application, including: 1) imaging with a device different from the one that will be used in operation (and which provides superior image quality). Note that this device does not necessarily need to be an ultrasound device, it could be of a different modality: e.g., CT, MRI, OCT, etc.; 2) enhancing High-Quality Images with an algorithm that would involve computational requirements that would be infeasible in actual operation of the device in its intended application; or 3) imaging with scanning parameters or system configuration that would involve excessive power, size, or cost requirements that could not be replicated in actual operation of the device in its intended application.

### Scanning Procedures

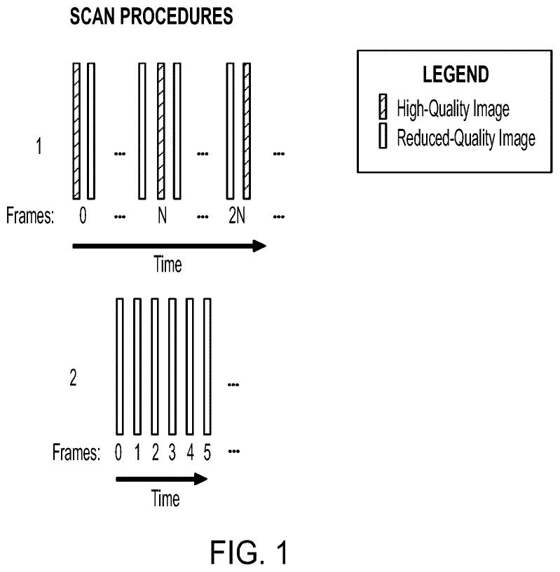

We consider two scanning procedures, illustrated in FIG. 1. In Scan Procedure 1, the ultrasound device is programmed to acquire data such that every Nth frame of the acquired ultrasound image sequence is a High-Quality Image, with the rest of the frames being Reduced-Quality Images. We will refer to the every Nth frame as an “Anchor Frame.” In Scan Procedure 2, the ultrasound device is programmed so that every frame of the acquired ultrasound image sequence is a Reduced-Quality Image. A wide range of image sizes/qualities are alternatively suitable, depending on what image sizes/qualities the ultrasound device is capable of providing or configures to provide.

### Using a Machine Learning Model to Recover Image Quality

The subject matter described herein comprises the acquisition of ultrasound image frames according to Scan Procedure 1 or Scan Procedure 2, followed by application of a Model to predict Restored Images based on the acquired images. Thus, one or more Restored Images comprise the Predicted Label, and one or more acquired images comprise the Input to the Model.

In one embodiment, a Model is designed to restore each Reduced-Quality Image in Scan Procedure 1 by predicting a Restored Image based on a Reduced-Quality Image and the two Anchor Frames that are nearest to it in the sequence (closest in time).

In another embodiment, a Model is designed to restore each Reduced-Quality Image by predicting a Restored Image based on frames from farther in the past (not closest in time). In such embodiments, the past frames optionally include: one or more previous high-quality frames, one or more previous Reduced-Quality frames, and even one or more frames produced earlier in the sequence by the processing methods described herein, and combinations thereof.

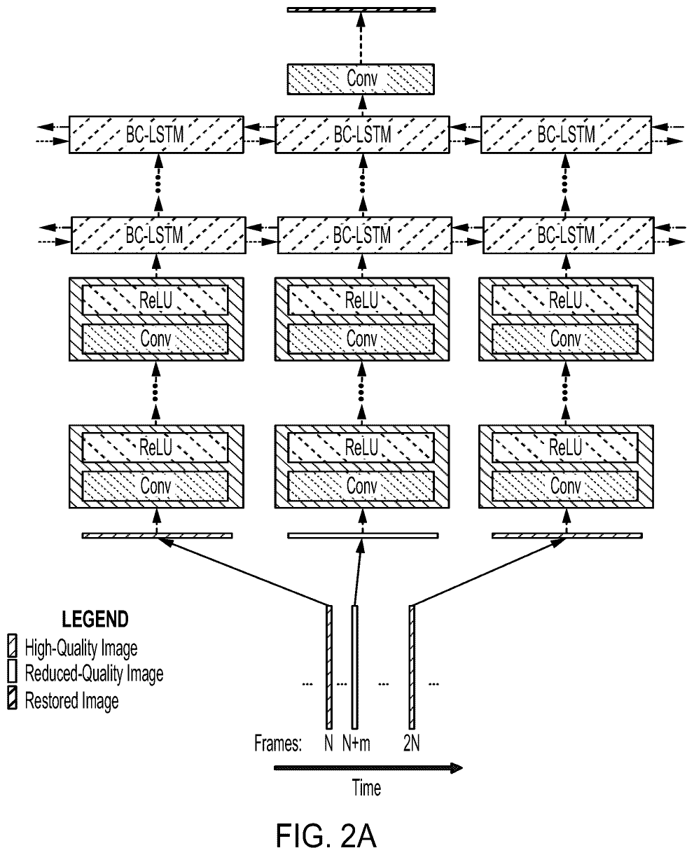

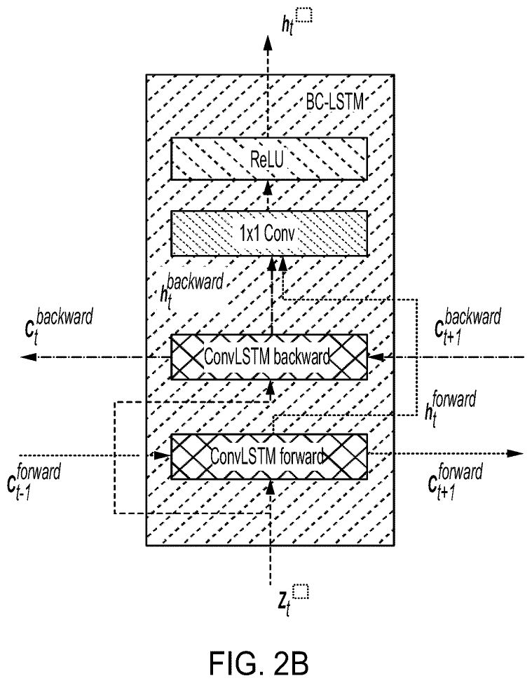

An exemplary Model to predict a Restored Image can be constructed using the deep learning architecture shown in FIG. 2A (with detail provided in FIG. 2B), comprising convolutional neural network (Conv) layers and bidirectional, convolutional, long short-term memory (BC-LSTM) layers. In this embodiment, the purpose of the Conv and ReLU layers is to capture and analyze spatial features of the image frames. The initial Conv layers have the effect of determining spatial features of the image. The BC-LSTM layers use information from the two Anchor Frames to assist in restoring the Reduced-Quality Image. In this embodiment, the BC-LSTM exploits temporal relationships among the image frames. The final Conv layer (uppermost in FIG. 2A) aggregates the outputs of the BC-LSTM modules to form the Restored Image.

In FIG. 2B, the “ConvLSTM backward” modules within all the “BC-LSTM modules” share the same parameters, whereas the Conv layers do not necessarily share common parameter values.

In this embodiment, training of the Model is accomplished by using two Anchor Frames and one Reduced-Quality Image to form the Input (as seen in FIG. 2A), with the True Label being a High-Quality Image or Enhanced-Quality Image depicting the same image content as the Reduced-Quality Image that the Model seeks to restore. In operation, the Model produces a Predicted Label comprising the Restored Image.

In a variation of this embodiment, the Model uses a Reduced-Quality Image and only the most recently acquired Anchor Frame to form the Input, with the Predicted Label comprising a Restored Image. This approach has the benefit of reliance on causal processing (i.e., it does not utilize images acquired after the Reduced-Quality Image), and thus avoids latency (delay) in producing the images. However, this approach has the disadvantage that it only exploits one Anchor Frame.

In a further variation of this embodiment, the Model uses all the acquired images in a sliding time window of length M to form the Input, with the Output comprising Restored Images corresponding to all the acquired images in the time window. If M=N, then every time window of acquired images contains one Anchor Frame and N−1 Reduced-Quality Images. In this instance, it may be beneficial to introduce an input variable to the Model to identify which of the acquired images is the Anchor Frame.

In a further variation of this embodiment, the Model uses unidirectional temporal processing, such as unidirectional convolutional LSTM, working only in the forward time direction.

In a further variation of this embodiment, the Model processes one image at a time, either continuously updating itself based on past information, or processing each image independently.

In a further variation of this embodiment, Scan Procedure 2 is used to acquire the data. In this case, the Model is trained by using one or more Reduced-Quality Images to construct the Inputs, and one or more corresponding High-Quality Images or Enhanced-Quality Images to construct the True Labels.

In each of these embodiments, the temporal processing provided by LSTM can be replaced by a convolutional neural network. This network can exhibit long-term memory of the image content seen in the image sequence if the temporal convolutions included in the model are dilated convolutions. Alternatively, the spatial and temporal processing can be merged within a unified spatio-temporal convolutional neural network architecture.

In each of these embodiments, the Model can be trained to predict either a High-Quality Image or an Enhanced-Quality Image by appropriate construction of the Training Set.

In each of these embodiments, the Model can be used to improve the quality of the Anchor Frames by using Enhanced-Quality Images as the Labels in the Training Set.

An alternative to the embodiment illustrated in FIG. 2A, still within the scope of the subject matter provided herein, is based on three-dimensional convolutional neural network layers (e.g., no BC-LSTM), with two of these three dimensions being spatial coordinates, and the third dimension being the time coordinate. In this alternative embodiment, the temporal nature of the image sequence is captured by this third dimension.

Power Saving Methods that Impact Image Quality

### Reduction in Receive Channel Count

In some embodiments, the front-end system hardware can perform analog signal conditioning, digitization, demodulation, decimation, beamforming, etc. One way of reducing the power consumed by the front-end hardware is to reduce the number of receive channels that are being processed. This manifests itself as a loss of spatial resolution, contrast resolution, and signal-to-noise ratio (SNR). In one case where a smaller aperture is used on receive (64 elements vs. 128 elements) the spatial resolution is decreased by a factor of 2 in the azimuthal dimension. In another embodiment, the number of receive elements is reduced by using the even or odd elements. This method maintains azimuthal spatial resolution (same effective aperture size), but sidelobes and grating lobes induced by the poorer spatial sampling decrease the contrast resolution of the resulting image.

It is also worth mentioning that in systems where fixed element pitch results in a steering limitations at higher frequencies due to grating lobes and other spatial sampling artifacts that such an approach could be used to regain image quality. In such an embodiment, one could synthesize virtual elements to achieve a higher spatial sampling rate at the expense of increased computation and power for the “high quality” frame and then use the normal aperture for the low quality frames.

### Reduction in Temporal Sampling Frequency

Reducing the temporal sampling frequency of the front-end ADC also reduces power. In an extreme case, reducing the sampling frequency causes temporal aliasing of the ultrasound signal and reduces image contrast and resolution.

### Reduction in Phase/Delay Quantization During Beamforming

Reducing the computational complexity of the beamforming process such as reducing the group delay quantization levels or not performing the fine phase alignment reduces processing power. However, this imperfect focusing scheme reduces image quality by deteriorating the point spread function (spatial resolution and contrast resolution).

### Reduction in the Number of Transmissions

In this method to reduce power, the number of transmissions is reduced by increasing the transmit line spacing and/or increasing transmit f/# by restricting the transmit aperture. The overall effect is reducing the number of transmissions and hence receiving processing lines that need to be computed per frame. The effect of this approach is a reduction in image SNR, spatial resolution and image contrast.

Another effect of reducing the number of transmissions would be to increase the number of parallel receive beams processed per transmission. However, forming more receive beams per transmission results in multibeam, block artifacts that are undesirable in terms of image quality. The approaches outlined in this disclosure could be used to address these undesirable imaging artifacts.

Included in this method would be cases where multiple focused transmissions (different transmit focal depths) or retrospective transmit/receive aperture synthesis would be used in order to create a high quality image frame.

### Reduction in Transmitted Power

Transmitting at a reduced transmit power (voltage level, number of transmit cycles, weaker focus/plane waves/diverging waves) decreases the SNR. In this case the “high quality” frames would be transmitted at higher voltage levels, perhaps using coded excitation, and/or pulse width modulation, which would have the added benefit of reducing pulse ringdown.

### Reduction in Image Processing Complexity

In one embodiment, the Reduced-Quality Image frames are not processed the same way as the High-Quality Image frames in terms of the complexity of the image processing algorithms that are applied (e.g., phase aberration correction, adaptive imaging, aperture coherence function-based imaging techniques, speckle reduction, edge enhancement, etc.). This naturally reduces the spatial resolution, contrast resolution, and potentially SNR in the output image.

While preferred embodiments of the present subject matter have been shown and described herein, it will be obvious to those skilled in the art that such embodiments are provided by way of example only. Numerous variations, changes, and substitutions will now occur to those skilled in the art without departing from the described methods and devices. It should be understood that various alternatives to the embodiments of the subject matter described herein may be employed in practicing the described methods and systems.

What is claimed is:

1. A medical ultrasound imaging method comprising:
forming a plurality of frames of an ultrasound image sequence, each having a high image quality;
forming a lower-quality frame of the ultrasound image sequence with reduced image quality; and
improving the image quality of the lower-quality frame frame using the plurality of frames.
